(19) United States
(12) Patent Application Publication
(10) Pub. No.: US 2012/0096763 A1
(43) Pub. Date: Apr. 26, 2012

(54) METHOD OF FORMULATING A FUEL COMPOSITION FOR USE IN INTERNAL-COMBUSTION ENGINES

(75) Inventor: William H. Klausmeier, Eugene, OR (US)

(73) Assignee: WILLIAM H. KLAUSMEIER FAMILY TRUST, San Francisco, CA (US)

(21) Appl. No.: 13/217,171

(22) Filed: Aug. 24, 2011

Related U.S. Application Data
(63) Continuation of application No. 12/105,164, filed on Apr. 17, 2008, now abandoned.
(60) Provisional application No. 60/974,779, filed on Sep. 24, 2007.
(60) Provisional application No. 61/036,007, filed on Mar. 12, 2008.

(51) Int. Cl. C10L 1/32; C10L 3/00; C07C 67/08

(52) U.S. Cl. 44/302; 554/172; 554/173; 48/197 FM

(57) ABSTRACT

A method of formulating a fuel composition for use in internal-combustion engines includes the step of providing a fuel. In addition, there is the step of forming the fuel composition by adding to the fuel a polar fluid and an emulsifier. The emulsifier is present in an amount effective for the biodiesel fuel, alcohol, water, and emulsifier to form an emulsion. The emulsifier includes an active surfactant mixture of oleic acid and ammonium oleate, wherein the ratio of oleic acid to ammonium oleate is no more than 60:40.

### CROSS-REFERENCE TO RELATED APPLICATIONS

This application is a continuation of U.S. patent application Ser. No. 12/105,164, filed Apr. 17, 2008 and entitled “Method of Formulating a Fuel Composition for Use in Internal-Combustion Engines”, which application claims priority to U.S. Provisional Patent Application Ser. No. 60/974,779, filed Sep. 24, 2007 and entitled “Microemulsion Fuel Compositions and Methods For Producing The Same” and also claims priority to U.S. Provisional Patent Application Ser. No. 61/036,007, filed Mar. 12, 2008 and entitled “Fuel Compositions for Use In Internal-Combustion Engines And Methods of Forming Using Such Compositions”.

### TECHNICAL FIELD

Fuel compositions, and particularly, diesel fuel compositions.

### BRIEF DESCRIPTION OF THE DRAWINGS

FIG. 1 is a schematic drawing that shows a new method for synthesizing monoglycerides.

FIG. 2 is a schematic drawing that shows how the method of FIG. 1 can be used to synthesize glyceryl-α-monooleate (α-GMO).

FIG. 3 shows apparatus for performing the methods shown in FIGS. 1 and 2.

FIG. 4 is a schematic drawing that shows how the partitioning method may be used to obtain high purity GMO industrially.

FIG. 5, graphically depicts how the surfactant molecules are oriented so that their hydrophilic, or “water-loving,” ends point inward, mixing with the water/ethanol phase, and their hydrophobic, or “water-hating,” ends point outward, mixing with the similarly hydrophobic constituents of the diesel fuel.

FIG. 6 is a schematic illustration of the qualities of various formulations of diesel fuel, ethanol and GMO.

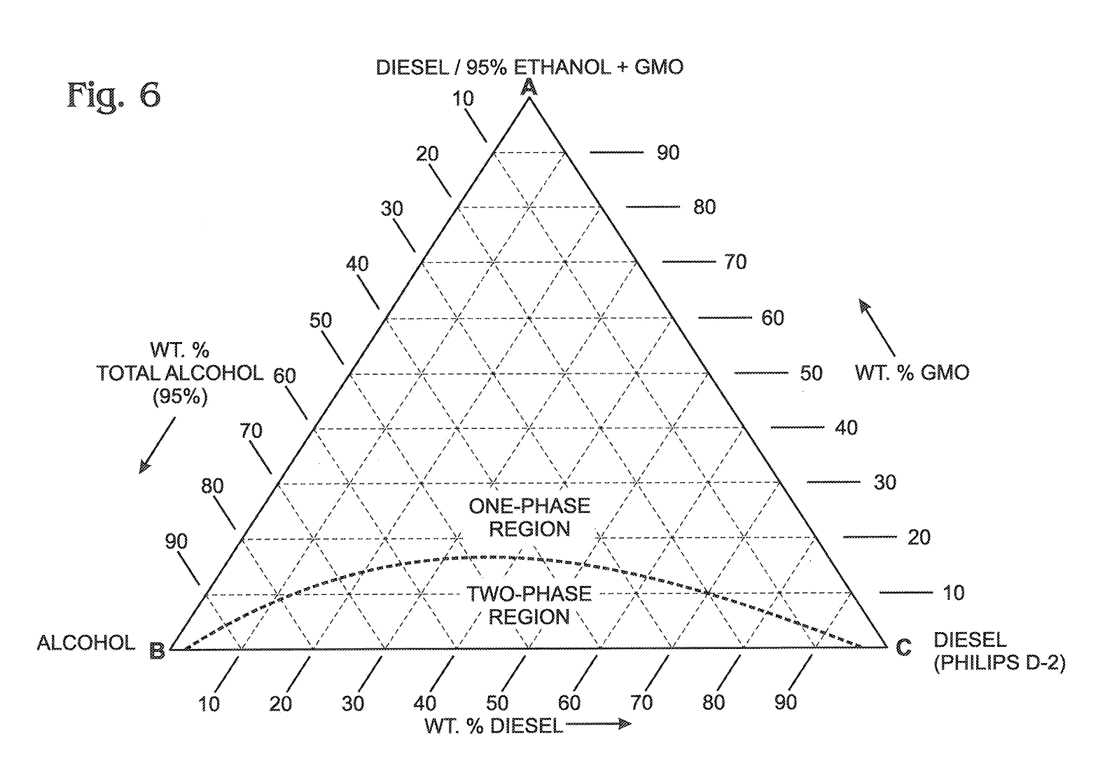

### DETAILED DESCRIPTION OF THE INVENTION

The invention provides fuel compositions for use in internal-combustion engines, and methods of forming and using such compositions.

The fuel compositions generally comprise (1) a hydrocarbon fuel, such as diesel, (2) a polar fluid, such as alcohol, water, and/or other oxygen rich fluids, (3) an emulsifier present in an amount effective for the hydrocarbon fuel, polar fluid, and emulsifier to form an emulsion; and (4) a cetane enhancer, such as 2-ethylhexyl nitrate. The emulsifier may be selected from a group consisting of noncyclic polyol fatty acid esters and noncyclic polyol fatty alcohol ethers. In some embodiments, at least about half of the emulsifier is selected from this group. In other embodiments, at least about half of this group is mono-substituted. The emulsifier also may consist essentially of a single molecular species having both polar and nonpolar portions.

The methods generally comprise methods of forming and using the fuel compositions, including components thereof. For example, the invention provides methods of forming the emulsifier, by synthesizing and/or purifying components of the emulsifier. These components may include noncyclic polyol fatty acid esters and noncyclic polyol fatty alcohol ethers.

These and other aspects of the invention are described in the following four sections: (1) synthesis of noncyclic polyol fatty acid esters and noncyclic polyol fatty alcohol ethers, (2) purification of noncyclic polyol fatty acid esters and noncyclic polyol fatty alcohol ethers, (3) fuel compositions, and (4) examples.

Monoglycerides of fatty acids have been used for years as surfactants in a variety of food, cosmetic, and other formulated products. In most applications, industrial-grade monoglyceride compositions having 40-55% monoglyceride content have proven suitable. However, the present application in fuel formulations requires high-purity monoglycerides to yield optimal performance, and inexpensive monoglycerides to be economically practical.

Monoglycerides have been synthesized by a variety of methods. Unfortunately, these methods generally yield products that must be further distilled or extracted to obtain high-purity monoglycerides. Moreover, these methods generally are unsuitable for forming monoglycerides of unsaturated fatty acids, such as oleic acid, because of oxidative decomposition at the point of unsaturation. U.S. Pat. No. 2,022,493 to Christensen et al. discloses the conventional method for synthesizing monoglycerides, which involves the transesterification of triglycerides with glycerol and sodium hydroxide to form the monoglycerides. However, the product of this method is a mixture of 40-55% monoglyceride, 20-30% diglyceride, and a remainder of unreacted triglyceride. U.S. Pat. Nos. 2,132,437 to Richardson et al. and 2,073,797 to Hilditch et al. disclose two methods of increasing monoglyceride selectivity by converting the triglyceride to free fatty acid before esterification. However, the products of these methods are still contaminated with at least 20% di- and triglyceride, and the methods are considerably more complex than the conventional method. U.S. Pat. No. 5,153,126 to Schroder et al. discloses a method for making additional gains in selectivity by using a lipase enzyme as the transesterification catalyst. However, this method is very costly and difficult to scale up.

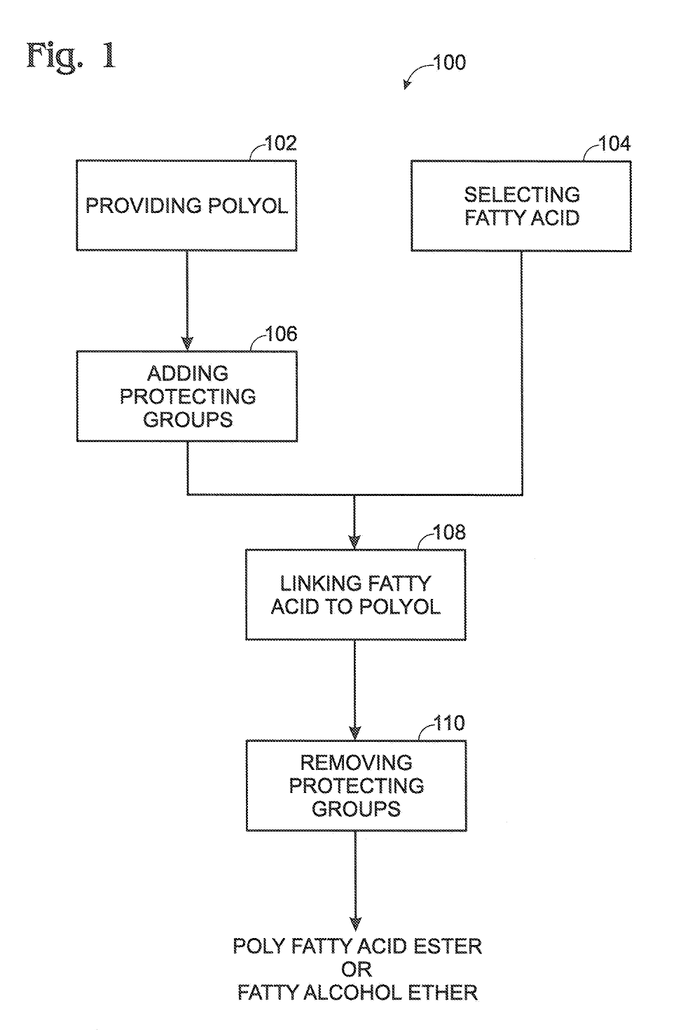

FIG. 1 shows a new method 100 for synthesizing monoglycerides, as well as other polyol fatty acid esters and polyol fatty alcohol ethers. Here, polyols are polyhydric alcohols, or alcohols having three or more hydroxyl (OH) groups. Examples of polyols include glycerol, which has three hydroxyl groups, and sugar alcohols, which generally have four to seven hydroxyl groups. A first step 102 in the method involves providing a polyol having at least three reactive alcohol groups. A second step 104 involves selecting a fatty acid or fatty chloride to react with the polyol. A third step 106 involves protecting all but a preselected one of the reactive alcohol groups on the polyol by reacting all but the preselected one of the reactive alcohol groups with protecting groups. A fourth step 108 involves linking the fatty acid to the polyol through an ester linkage or linking the fatty chloride to the polyol through an ether linkage by reacting the fatty acid or fatty chloride with the preselected one of the reactive alcohol groups. A fifth step 110 involves forming the polyol fatty acid ester or forming the fatty alcohol ether by removing the protecting groups. The first, second, and third steps may be performed in any order, as long as the third step follows the first step.

These steps may be performed under conditions that would tend not to substantially reduce an unsaturated fatty acid or fatty chloride. Such conditions may include performing one or more of the steps in an inert atmosphere, such as a nitrogen atmosphere, or performing one or more of the steps in the absence of light.

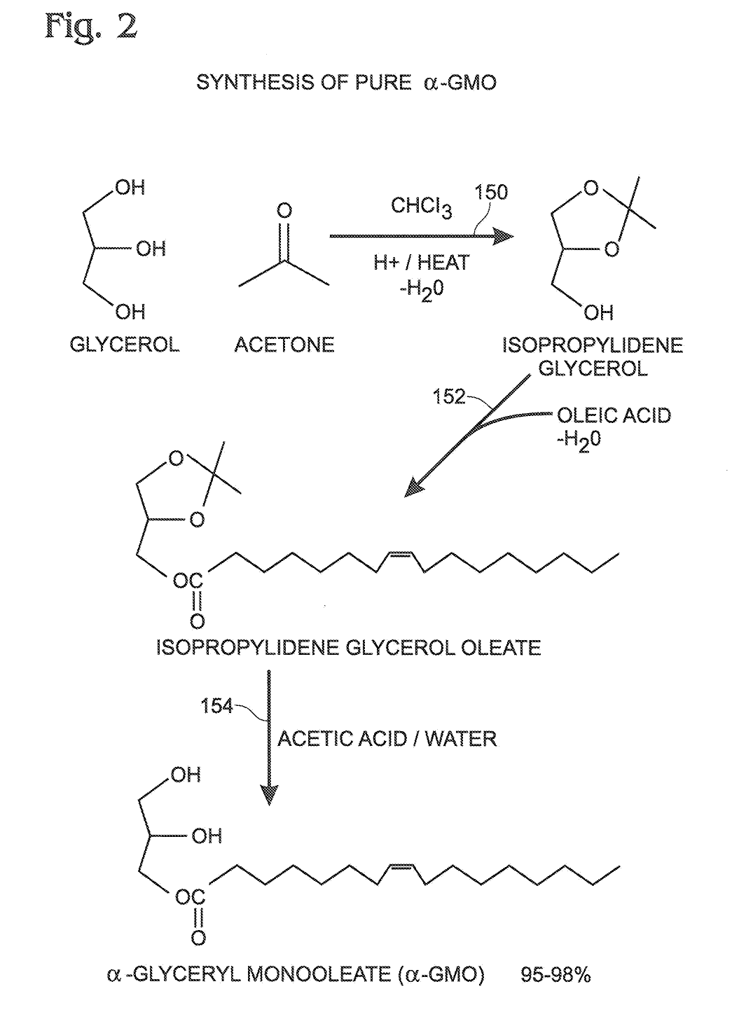

FIG. 2 shows how the method of FIG. 1 can be used to synthesize glyceryl-α-monooleate (α-GMO). Here, the polyol is glycerol, the fatty acid is oleic acid, and the protecting group is derived from acetone. In a first step 150, the glycerol is reacted with the acetone in the presence of an acid catalyst to form an intermediate acetonide, 1,2-iso-propylidene glycerol. The preferred acid catalyst is p-toluenesulfonic acid, but any concentrated mineral acid will suffice. Suitable solvents include any solvent that (1) does not react with the reactants, (2) is easily separated from acetone in a fractionating column, and (3) will carry water over by vapor condensation. Such solvents include benzene and solvents having 1-2 parts of chlorocarbons, such as chloroform. In a second step 152, the 1,2-iso-propylidene glycerol is reacted with oleic acid to form the corresponding 1,2-iso-propylidene glyceryl ester. In a third step 154, the 1,2-iso-propylidene glyceryl ester is reacted with aqueous acetic acid to remove the protecting group and give the corresponding α-monoglyceride. Acetic acid acts as both solvent and acid catalyst. Water is added at a rate that sustains hydrolysis without rendering reactants insoluble. Hydrolysis also can be effected by formation of intermediate borate esters, which are then hydrolyzed with water.

The methods in FIGS. 1 and 2 can be employed with a wide range of organic acids, requiring only slight modifications in product work-up. Generally, end-substituted α-polyols can be synthesized from odd-numbered polyols of the formula CH₂OH(CHOH)ₙCH₂OH (n=1, 3, 5 . . . ) by forming the protecting group using acetone, among others, as described above. Alternatively, inside-substituted β-polyols can be synthesized by forming the protecting group using benzaldehyde, among others. In FIG. 2, the glycerol would react with benzaldehyde to form 1,3-benzylidene glycerol, which would yield a corresponding β-glyceryl ester upon esterification and removal of the benzylidene group by catalytic hydrogenation.

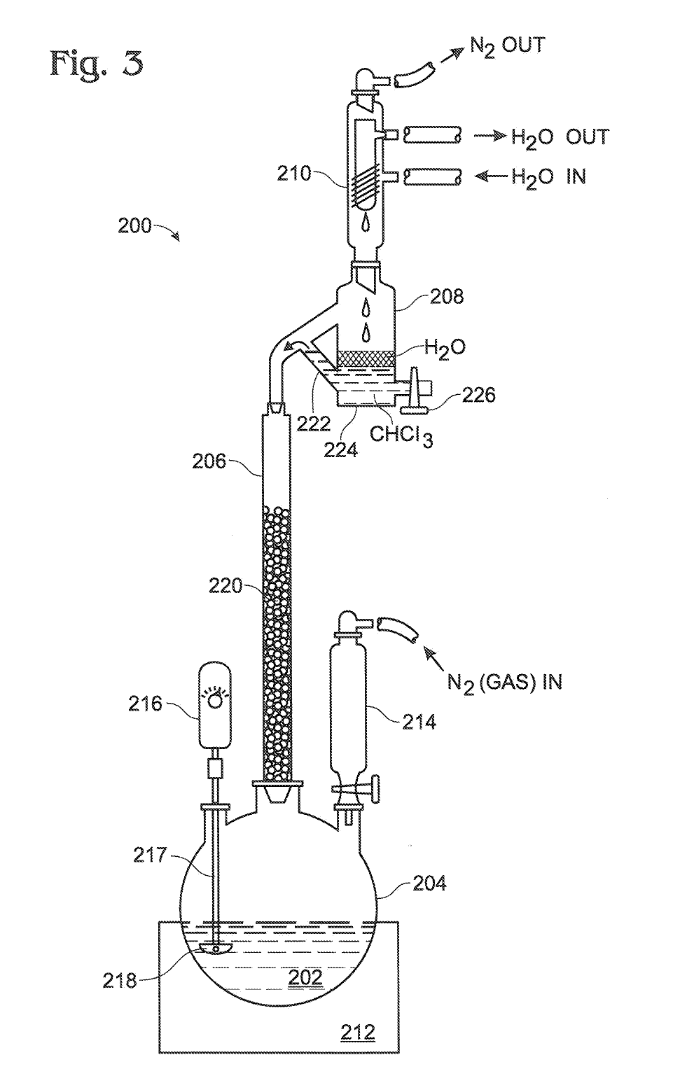

FIG. 3 shows an apparatus 200 for performing the methods shown in FIGS. 1 and 2. A solution 202 of acetone, glycerol, and acid catalyst in chloroform is placed in a flask 204 such as a 3-neck round-bottom flask fitted with a fractionating column 206, light-oil separating trap 208, and condenser 210. The flask may be placed in a heating mantle 212 and further fitted with a dropping funnel 214 and a stirrer motor 216 configured to drive a stir rod 217 and paddle 218. The reaction mixture is refluxed, and water is collected in the trap until no more water forms.

The desired organic acid is then added to the reaction mixture, and reflux is continued until no additional water collects in the trap. The solvent is distilled off the reaction mixture, and the residue is dissolved in glacial acetic acid and heated at 60° C. for several hours as water is gradually added. The reaction product precipitates upon cooling and dilution with additional water.

The apparatus is a simple and efficient means of driving the reaction to completion. During reflux, chloroform and water vapors are separated from reactants by the use of fractionating column 206, which is packed with glass beads 220. The chloroform/water vapors are then condensed by means of condenser 210, such as a Friedrichs condenser, with the condensate flowing down into light oil-type separation trap 208, where the water and chloroform phases separate. The denser chloroform phase continuously returns to the reaction vessel via a sidearm 222, while water accumulates in a receiver trap 224. Water can be periodically removed from the receiver trap via a stopcock 226 if the production scale exceeds the volume capacity of the trap. Upon completion of step 1, the desired organic acid can be added and step 2 then carried out without interruption of reflux. The system works smoothly with little operator attention up to semi-pilot (22-liter reaction volume) scale. Although reaction times for steps 1 and 2 were fairly long (24-26 h) with the equipment used, reaction times can be shortened greatly by increasing the capacity of the fractionating column and condenser. Chloroform solvent can be replaced with other less harmful solvents, as long as the substitute has a density greater than water and an appropriate boiling point.

The final acid hydrolysis step using glacial acetic acid represents an improvement over other acetonide hydrolysis reagents previously employed, such as mineral acids or boric acid/2-methoxyethanol. The process takes advantage of the product's limited solubility in aqueous acetic acid. By adding water only gradually during hydrolysis, all reactants are kept in solution throughout the step. Once hydrolysis is complete, the addition of a small amount of water to the cooled product solution causes the product to precipitate. The acetic acid/water mixture, containing less than 20% water, is then decanted and can be purified and recycled. The combined attributes of selectivity, simplicity, and recyclability of materials all make the process amenable for use at an industrial scale. In contrast, in the past, the acetonide protecting group was removed using a two-step process. The acetonide was first converted into the borate ester using boric acid and 2-methoxyethanol, and the borate ester was then extracted into ether and washed with water to hydrolyze the ether back to the original diol functionality. This procedure is cumbersome, and some of the reagents are too costly to use on an industrial scale.

There are many attributes of the present process that render it a practical means for monoglyceride production. All three steps of the reaction sequence are accomplished in the same reaction vessel. By using a co-solvent such as chloroform in combination with a separation trap, water is continuously removed from the reaction mixture, thereby driving both the acetonide and ester formation steps to completion. In the past, acetonide formation steps were driven to completion by mechanisms only suitable at very small scales, such as using water carrier solvents such as chloroform or benzene, and either a collection tube or Soxhlet extractor filled with drying agent to remove water from the reaction mixture as it is formed. The solvent mixture obtained by distillation of the iso-propylidene glyceryl ester product mixture can be recycled for use in the next batch. The final acetonide hydrolysis step is mild and fast, and the acetic acid recovered can be purified and recycled.

The product yield from each step is virtually quantitative, and the overall yields range from 94-98%. Thin layer chromatography (TLC) reveals only traces of residual reactants and no di- or triglyceride contaminants. The product work-up is easy; involving neutralization of residual acid with sodium bicarbonate, followed by three water washes. This crude product can be used in microemulsion formulations without further purification. However, partitioning of the crude product between aqueous ethanol and hexane removes residual reactants; concentration of the aqueous ethanol phase affords a pure α-monoglyceride product that readily crystallizes. In microemulsion formulations using glyceryl-α-monooleate (α-GMO) as the surfactant, only one-sixth the amount of this α-monoglyceride is needed versus the amount of industrial grade glyceryl monooleate (GMOI) needed otherwise to emulsify an equivalent amount of aqueous ethanol in diesel fuel.

The method also may be used to synthesize glyceryl fatty alcohol ethers from the corresponding fatty alcohol chloride. For example, the method may be used to synthesize 1,2-iso-propylidene glyceryl R, where R is a hydrocarbon chain, from RCI and 1,2-iso-propylidene glycerol.

The monoglyceride product may be purified by a variety of methods. U.S. Pat. No. 3,826,720 to Lowrey discloses a monoglyceride purification method based on the partitioning of crude glyceride mixtures between aqueous methanol and hexane. Monoglycerides preferentially migrate to the aqueous methanol phase. However evaporation of the aqueous methanol proves difficult because of excessive foaming. Since the present application uses the monoglyceride in combination with aqueous ethanol, it would be advantageous if aqueous ethanol could be substituted for aqueous methanol in an analogous procedure. Working with product solutions would reduce the materials handling problems associated with such products, which are typically very tacky and viscous in the liquid state. Such a method might also be useful for upgrading industrial grade monoglycerides.

The solvent partitioning purification method for removing residual contaminants from the crude product is also effective for upgrading the purity of industrial grade GMOI. It is based on a commonly employed method using a counter-current separatory column with aqueous methanol as the descending phase and hexane as the ascending phase to separate monoglycerides from di- and triglyceride contaminants. The monoglycerides migrate to the aqueous methanol phase, while the di- and triglycerides migrate to the hexane phase. In the present application, 5% aqueous ethanol was substituted for the aqueous methanol. The crude GMO sample is dissolved in 10 parts of hexane and 15 parts of 5% ethanol to afford a homogeneous solution. Upon addition of 1 part of water, the solution separates into two phases. Concentration of the aqueous ethanol phase affords a viscous oil that crystallizes on standing and contains very little residual di- and triglyceride by TLC. Concentration of the hexane phase affords an oil that is primarily di- and triglyceride by TLC.

In formulation experiments with diesel fuel, the GMO thus purified performs as well as crude α-GMO. If either this product or crude α-GMO is again partitioned by the same procedure, the requirement for either surfactant is further reduced by 50%, which represents an overall six fold reduction in GMO requirement compared to industrial GMOI. Further partitioning does not afford significant additional performance improvements. This method therefore appears to be effective in removing both residual reactants and di- and triglycerides from monoglyceride products.

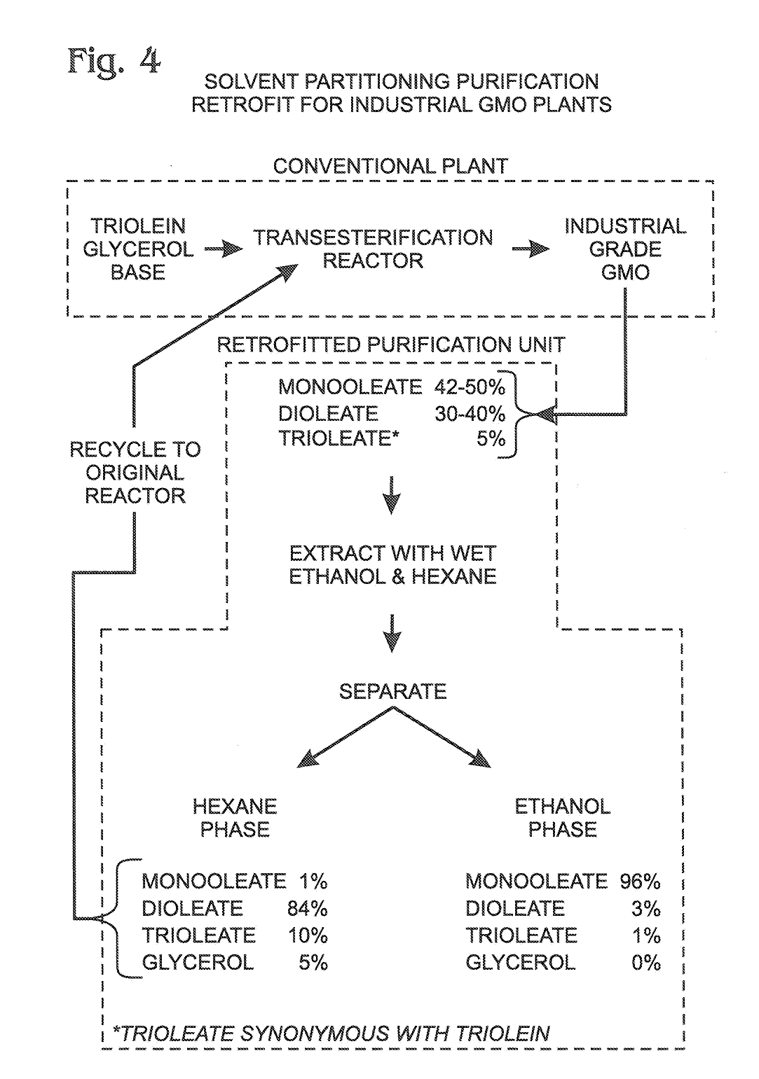

FIG. 4 shows how the partitioning method may be used to obtain high purity GMO industrially. Existing GMOI plants could be retrofitted with such an extraction purification system to enable them to produce high purity GMO without greatly increasing manufacturing costs. The di- and triglyceride mixture isolated from the hexane fraction could be recycled to the original transesterification reactor. Also, since the GMO product is used in combination with aqueous ethanol in diesel microemulsions, the aqueous ethanol does not have to be completely removed for use in fuel formulations. This would greatly simplify materials handling.

### Fuel Compositions

One purpose of the synthesis and purification research is to provide options for the low cost manufacture of purified GMO. The ability to accomplish this has proven critical to the feasibility of using GMO and other polyol fatty acid esters as surfactants for stabilizing water/ethanol/diesel microemulsions. Previous investigations have demonstrated that such microemulsions can be made using industrial grade GMOI but have serious drawbacks. Nearly three parts of GMOI are needed to create a 10% microemulsion of 5% aqueous ethanol with diesel fuel that is stable at room temperature. At current prices, the cost of a 30 wt % GMOI: 10 wt % aqueous ethanol: 60 wt % diesel is more than $2.50/gallon which is more than twice the current price of diesel. Such emulsions also are temperature sensitive, and prolonged storage at temperatures below the freezing point of water results in the precipitation of solids and/or phase separation depending upon the particular source of GMOI. The composition of GMOI varies considerably from supplier to supplier, making it difficult to predict the behavior of a particular source of GMOI.

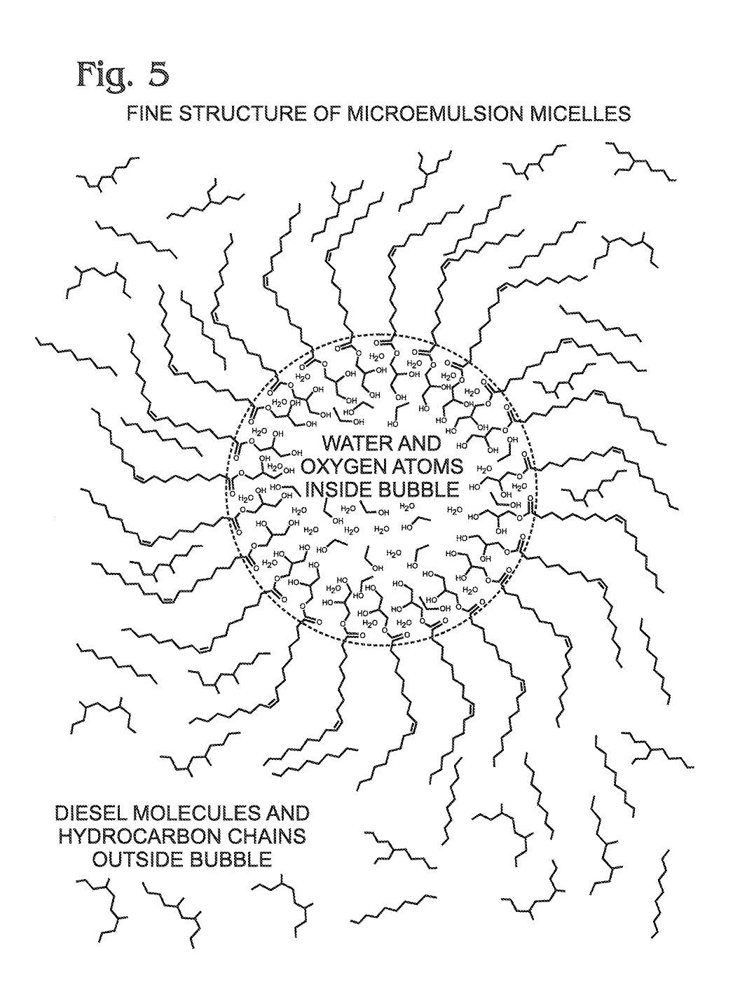

These microemulsions are considered to be extremely fine colloidal dispersions consisting of micelles, or “bubbles,” of water and alcohol coated with a layer of surfactant. As depicted in FIG. 5, the surfactant molecules are oriented so that their hydrophilic, or “water-loving,” ends point inward, mixing with the water/ethanol phase, and their hydrophobic, or “water-hating,” ends point outward, mixing with the similarly hydrophobic constituents of the diesel fuel. This is how the surfactant draws these two incompatible phases together into stable microsuspensions of tiny water/ethanol bubbles dispersed throughout a diesel oil phase. There is an excellent mechanistic role for GMO and other fatty acid monoglycerides to disperse and form a uniform coating around these bubbles, thereby rendering the emulsion stable.

However, the industrial grade product, GMOI, is only 40-55% monooleate with the balance being di- and trioleate. Neither the di- or trioleate fits well into the model; the dioleate has little hydrophilic character and the trioleate none. Their presence only serves to interfere with the action of the monooleate. The need for higher purity material drove the investigations into synthesis and purification options. It was subsequently discovered that a higher purity grade of GMO, which analyzed as 90% monooleate, is commercially available for specialized uses in cosmetics. Formulation tests using either the α-GMO obtained by direct synthesis or the 90% GMO available commercially demonstrated a six-fold reduction in the amount of GMO needed. The 30:10:60 GMOI: aqueous ethanol: diesel fuel formulation possible with the industrial grade product could be achieved using a 5:10:85 α-GMO (or 90% GMO): aqueous ethanol: diesel fuel formulation with high purity GMO (α-GMO and 90% GMO). The fact that a six-fold increase in effect was achieved with only a two-fold increase in purity has important implications. The disproportionate increase suggests that the relationship between the constituents is, indeed, quite specific. The dilution effects of the contaminants are compounded by another effect, which is most likely their interference in the efficient ordering of the monooleate molecules. Microemulsions made with high purity GMO exhibit the positive Tyndall effect expected of colloidal dispersions. All indications support a well-ordered micelle with an ethanol/water core and a monomolecular layer of monooleate molecules.

It also was found that the formulations using high purity GMO had much more thermal stability. In addition to the known antifreeze action of the ethanol, it is reasonable that the hydrophilic ends of the monooleate molecule duplicate the antifreeze ethylene glycol and thereby cause an analogous effect. The addition of a small amount of high purity GMO considerably enhances the microemulsion's thermal stability. Whereas microemulsions using industrial GMOI were only stable for a period of hours at −10° C., microemulsions using high purity GMO could be stored at −10° C. for months without phase separation or the formation of any precipitate. The additional stabilizing benefits of adding small portions of ethylene glycol, iso-propanol, or a 6:1 mixture of cyclohexanol and cyclohexanone also were noted, with small portions of either (less than 0.5%) further stabilizing the emulsions down to −20° C. For practical purposes, the high purity GMO enables use of the fuel without concern in most of the coastal and southern United States. Fuel system heaters that might be needed in cold climates are already in use for diesel trucks operating in these regions.

It is possible that there may be some cold starting difficulties, because formulations with diesel incorporating high levels of alcohol have exhibited such problems in the past. Also, engine timing in diesel engines varies with engine type and model year, and this can affect the emissions reductions achieved. Although water has a beneficial effect by lowering combustion temperature, it also can retard ignition, which can have a counter-productive effect depending upon engine timing. If either of these problems arises with particular formulations, the addition of cetane enhancing organic nitrates such as 2-ethyl hexyl nitrate or organic peroxides such as ditertiary butyl peroxide should alleviate either problem. Although the presence of nitrogenous components in emulsion formulations may contribute to NOₓ formation, there is strong evidence that the nitro groups in alkyl nitrate cetane enhancers are converted to harmless nitrogen gas in the combustion process. However, the surfactant has a good cetane value itself, so the levels of cetane enhancer that may be required would not be high (0.5-3.0 wt %) in any event. 2-Ethylhexyl nitrate also was found to have a modest stabilizing effect in emulsion formulations.

The microemulsions using high purity GMO also tolerate the presence of more water than that present in just the 5% aqueous ethanol phase, as long as the ethanol content is relatively high. Formulations using a 5:10:85 ratio can tolerate up to two percent added water. Stable formulations with water contents exceeding 5% have been made using only 2 parts high purity GMO per part of water. Thermal stability is compromised as the water content is increased, but this, too, can be compensated for by increasing the ethanol or GMO content or by using the stabilizing additives previously noted. The presence of water accounts for NOₓ-reducing effects of microemulsions by reducing combustion temperature and results in smoother running by broadening the temperature-time profile. The particulate reduction effects also are accounted for by the “steam explosion” of the microbubbles upon combustion, which better atomizes the fuel and thereby results in more complete combustion. The ability to control the level of water is important in efforts to find the maximum emissions-reducing effects. Ethanol also burns very cleanly in diesel engines, producing no smoke, so its presence can dramatically reduce particulate emissions. Ethanol also contributes to the moderation of combustion temperature and can, thereby, reduce NOₓ emissions by 10% or more even in the absence of any water.

Another constituent, ammonia, shows a dramatic NOₓ-reducing effect. Stable emulsions also can be made using GMO in combination with the ammonium salt of oleic acid or other suitable carboxylicacids. Ammonia is used to reduce NOₓ in exhaust gas in both high-temperature and catalytic low-temperature systems. It reacts with NOₓ to produce harmless nitrogen gas and water. It was reasoned that introducing ammonia in the form of ammonium oleate might neutralize NOₓ formed during the combustion process, and the emissions data presented at the end of the example section show a large NOₓ reduction when ammonia is present in this form. Ammonia reduces NOₓ emissions in formulations both with and without cetane enhancer. In formulations with cetane enhancer, ammonia also appears to reduce particulate emissions. Calculations show that 12-59% of the ammonia present is consumed in neutralizing NOₓ. Ammonia and oleic acid also are inexpensive and reduce the requirement for the more expensive GMO.

It is possible to combine almost any proportions of ingredients by using the appropriate amount of high-purity GMO surfactant. However, significant emissions reductions have been noted at a level of only 10% aqueous ethanol (overall water content of 0.5 wt %). Since the GMO costs more than diesel fuel, the quantity used should be kept to the minimum needed to obtain the desired effect. The estimated cost of a microemulsion containing 10 wt % aqueous ethanol is about 20% greater than diesel fuel alone at current diesel, ethanol, and high-purity GMO prices. Reasonable reductions in manufacturing costs could reduce the price differential to as little as 10% at the current, very low price for diesel. Only a modest increase in diesel price is needed to offset this disadvantage.

All constituents in the subject formulations come from renewable resources, the aqueous ethanol being produced by fermentation and the GMO being derived from corn oil. The microemulsion formulations that are the object of the present invention are fully renewable fuels, the 5:10:85 formulation having a renewable content of 15%. Users not only qualify for consideration as a renewable fuel but also may qualify for CO₂ reduction credits should programs to curb global warming be put into effect.

Although the GMO:aqueous ethanol:diesel fuel formulations have been identified as one preferred embodiment, the method has considerable generality. Stable emulsions can be formed with any of the C₁-C₄ alcohols. The level of monoglyceride required can be reduced through the use of the ammonium salts of fatty acids, preferably unsaturated fatty acids such as oleic acid. Monoglycerides incorporating other unsaturated fatty acids such as elaidic, erucic, or linoleic acid also are effective in amounts comparable to those of GMO and exhibit reasonable thermal stability. Monoglycerides incorporating saturated fatty acids such as lauric, myristic, or stearic acid also form microemulsions, but most are thermally unstable. To those skilled in the art, it is evident that both the synthesis and the application can be generalized to a wide range of polyol fatty acid esters and polyol fatty alcohol ethers. The corresponding glyceryl fatty alcohol ethers exercise effects comparable to their ester analogues. This is to be expected from the model because the position of the oxygen absent in the ethers has no bearing on the key structural features of the monoglycerides as surfactants.

It also should be noted that the use of these emissions-reducing microemulsions will enable the use of additional control methods such as catalytic conversion and exhaust gas recycle that are currently impractical because of the high level of particulate soot in diesel exhaust.

The following examples illustrate without limitation these and other aspects of the invention.

### EXAMPLES

### Example 1

### Direct Synthesis of α-GMO with Hydrolysis Via the Borate Ester

50.0 g (0.543 moles) of glycerol, technical grade was added to a 500 mL round bottom flask fitted with a magnetic stir bar, heating mantle, and 400 mm fractionation column packed two-thirds full with glass beads connected to a light oil separation trap and Liebig condenser, as shown in FIG. 3. 75 mL (59.1 g, 1.02 moles) of acetone, reagent grade and 100 mL chloroform, reagent grade and methanol-free, and 0.5 g (0.0029 moles) of p-toluenesulfonic acid, reagent grade were then added to the flask. The reaction mixture was heated to reflux, and condensate was collected in the trap with periodic removal of accumulated water from the trap via the stopcock. Reflux was maintained until no more water accumulated (approximately 4 hours with the water recovery rate being 4 mL/h and total water recovered being 10 mL). Reflux was interrupted, and the reaction mixture was allowed to cool 30 minutes, and then 50.0 g (0.177 moles) of oleic acid, technical grade was added. A nitrogen inlet/outlet was placed on top of the condenser, slow nitrogen flow was initiated, and reflux was resumed for four hours, collecting an additional 3.1 mL of reaction water. The reaction flask was shielded from light. The reaction mixture was allowed to cool, then 0.55 g (0.0067 moles) of anhydrous sodium acetate, technical grade was added, and the flask was capped and shaken vigorously. The product solution was transferred to a 500 mL separatory funnel and washed 4 times with 100 mL portions of distilled water. Solvent was removed from the product mixture with warming under mild vacuum to give 68.5 g (0.173 moles, 97.7% yield) of 1,2-iso-propylidene glyceryl oleate as a light amber liquid.

The crude ester product was placed in a 500 mL Erlenmeyer flask, and 200 mL 2-ethoxyethanol and 60.0 g (0.97 moles) of ground powdered boric acid, technical grade were added. The mixture was heated on a hot plate at 100° C. for 45 minutes then allowed to cool. The boric acid gradually dissolved upon heating but white solids, presumably unreacted boric acid, precipitated on cooling. The mixture was transferred to a 1 L separatory funnel and extracted with 500 mL of diethyl ether. The ethereal solution was washed 4 times with 500 mL portions of distilled water. The third and fourth water washes formed strong emulsions that took 45 minutes to break and partition. The ethereal solution was dried over anhydrous sodium sulfate, technical grade, filtered into a 1 L beaker, and gently warmed until all the ether was evaporated. The oil was placed in a vacuum desiccator and subjected to high vacuum overnight to give 44.6 g (0.125 moles, 72.3% yield) of light amber viscous oil that crystallized on standing. The product melting point was 32-37° C. Chromatographic analysis by comparison with known standards using silica gel plates in 10% methanol in benzene confirmed that the product was glyceryl-1-monooleate uncontaminated with any di- or trioleate with trace amounts of intermediate 1,2-iso-propylidene glycerol and 1,2-iso-propylidene glyceryl oleate impurities.

### Example 2

Scaled-Up Direct Synthesis of α-GMO Using Hydrolysis in Acetic Acid

To a 22 L 3-neck flask fitted with mechanical stirrer, heating mantle, 900 mm fractionating column two-thirds full of glass beads and fitted with a light oil-type liquid-liquid separator and Friedricks condenser and nitrogen inlet and outlet were added: 3,000 g (2,372 mL, 32.6 moles) glycerol, technical grade, 4,500 mL (3,546 g, 61.1 moles) acetone, reagent grade, 4,800 mL chloroform, Unisolv methanol-free grade, and 12.0 g (0.07 moles) p-toluenesulfonic acid, reagent grade. The reaction mixture was carefully heated to a state of reflux, producing an appropriate rate of condensation into the separator. Reflux was continued until no more water accumulated in the separator (approximately 24 h at a collection rate of 25 mL/h with 580 mL of water collected). The separator was designed with sufficient capacity (1 l) to eliminate any need to remove reaction water during reflux. Nitrogen gas flow was initiated, and the reaction vessel was protected from light. 3,070 g (3,431 mL, 10.87 moles) of oleic acid, technical grade were added to the still hot reaction mixture via a dropping funnel, and reflux was continued until no more water accumulated in the separator (approximately 20 h at a collection rate of 10 mL/h with 195 mL collected). Heat was discontinued, and 26.4 g (0.32 moles) of anhydrous sodium acetate, technical grade was added with vigorous stirring. After cooling, 4 L of distilled water was added and thoroughly mixed. Mixing was stopped, the phases were allowed to separate, and the aqueous phase was removed by siphon. This step was repeated twice with 8 L portions of distilled water.

The dense organic phase was separated from residual water in a separatory funnel and charged into a clean 22 L flask for distillation and hydrolysis. The flask was fitted with a distilling head and 900 mm Liebig condenser, heating mantle, and mechanical stirrer. Chloroform and residual acetone were distilled off. The distillation temperature went from 57° C. to 72° C., at which point 4,500 mL of distillate had been collected. The distillation system was put under mild vacuum, and another 300 mL of distillate were collected. In subsequent runs, the chloroform/acetone solvents were distilled entirely under mild vacuum, such that the head temperature was kept between 40-45° C. This reduced the distillation time to 2 h. 4,083 mL of glacial acetic acid, technical grade was added, and the reaction mixture was warmed to 60° C. 600 mL of distilled water was added until the reaction mixture just became cloudy. An additional 1,800 mL of distilled water were added in 100 mL portions via a dropping funnel whenever the reaction mixture completely cleared, and the temperature was maintained between 60-70° C. After 5 h, the reaction mixture was allowed to cool overnight. In subsequent runs, the distilled water was added as fast as the cloudiness dissipated, which reduced the reaction time to 2 h. The reaction mixture was poured into 12 L of rapidly stirring distilled water. Subsequent trials showed that the added water volume could be reduced to as little as 2 L without significantly affecting product purity or handling as long as the crude product mass was washed well. After the precipitated product mass had time to set on standing (going from a viscous liquid to a semi-solid state), the aqueous acetic acid was decanted off. The mass was washed 3 times with 6 L of distilled water with maceration to penetrate the product mass. The mass was transferred to a glass reactor and treated with 5 L of saturated aqueous sodium bicarbonate with warming and maceration. When effervescence subsided, the bicarbonate solution was decanted, and the mass was washed twice with 4 L of distilled water and then heated just to the boiling point in 6 L of fresh distilled water and allowed to cool gradually to give an amber gel which formed at the surface as the α-GMO melted and then re-solidified. This process effectively expresses most of the water from the mass. The gel was dried overnight under a strong vacuum with gentle warming at 45-50° C. to give 3,717 g (10.42 moles, 96% yield) of viscous amber oil that crystallized on cooling. The product melting point was 33-36° C. TLC analysis showed a single spot corresponding to glyceryl-1-monooleate (α-GMO) with no di- or trioleate contamination and only faint traces of acetonide intermediates.

### Example 3

### Purification of Industrial GMOI by Solvent Partitioning

5.0 g of GMOI (Canamex Glicepol 182 Lot G-20Z7) was weighed into a flask. 75 mL (50 g) hexane, technical grade and 94 mL (75 g) 5% aqueous ethanol, technical grade were added and the contents mixed until a uniform solution was obtained. An additional 7.0 g of distilled water was added to the flask and mixed and decanted into a 250 mL separatory funnel. The funnel was capped, thoroughly shaken, then allowed to stand so the phases could separate. The phases were separated into two 125 mL Erlenmeyer flasks, and the solvent was removed by gentle heating. The hexane fraction weighing 45.6 g with solvent afforded 2.96 g of light tan oil. The ethanolic phase weighing 86.39 g with solvent was evaporated, then 100 mL anhydrous ethanol was added and evaporated to remove any residual water to give 1.92 g of light tan oil that spontaneously crystallized on cooling. TLC analysis using silica gel plates in 10% methanol in benzene showed the hexane-derived oil to be primarily di- and trioleate with some residual monooleate and the ethanol-derived solid to be primarily monooleate with only traces of di- and trioleate evident.

### Example 4

### Hydrous Ethanol with Refined GMOI in Diesel Fuel

In formulation experiments using either unrefined or refined GMOI, 10 parts diesel fuel were mixed with 1 part hydrous ethanol in a flask, and then the GMOI sample was added in portions until a clear homogeneous mixture was obtained. The final proportions are compared in the following:

| | Wt % of Mixture | Wt % of Mixture |
|---|---|---|
| Component | using GMOI | Using Refined GMOI |
| Diesel fuel | 72.8 | 81.3 |
| Hydrous ethanol | 7.3 | 8.1 |
| GMO sample | 19.9 | 10.6 |

The formulation using the refined GMOI also appeared particularly stable to temperature, remaining completely clear on prolonged storage at −9° C. Refining reduced the amount of GMOI needed by 50%. This method when applied to crude α-GMO obtained by direct synthesis also afforded substantial performance improvements, which indicated that it also is effective in removing intermediate acetonide contaminants as well.

### Example 5

### Hydrous Ethanol with Industrial GMOI in Diesel Fuel

40.0 g of industrial grade glyceryl monooleate (GMOI) of 40%+monooleate content (PPG Industries) was blended with 60 g of diesel fuel until a homogeneous mixture was achieved. Hydrous ethanol (190 proof) was then added in portions and mixed until homogeneous. The mixture remained homogeneous over the following range of proportions:

| Component | Wt % of Mixture |
|---|---|
| GMOI | 25.0-39.2 |
| Hydrous ethanol | 2.0-37.5 |
| Diesel | 37.5-59.0 |

The mixtures with the above ranges were clear and stable at room temperature. The phases did not separate after refrigeration for 24 h at −12° C. until the ethanol concentration exceeded 25%. At room temperature up to 2.0 wt % water could be added before phase separation was noted.

To determine the minimum amount of GMOI needed to effect a stable emulsion of hydrous ethanol (190 proof) with diesel fuel, GMOI (PPG Industries), hydrous ethanol, and diesel fuel were mixed in the following proportions with the indicated results:

| Component (grams) | | | |
|---|---|---|---|
| Diesel Fuel | GMOI | Hydrous ethanol | Effect at Room Temperature |
| 80 | 10 | 10 | No emulsion, two distinct |
| | | | phases |
| 70 | 20 | 10 | Very cloudy, slow separation |
| | | | of phases |
| 65 | 25 | 10 | Cloudy, slow separation of |
| | | | phases |
| 60 | 30 | 10 | Clear stable emulsion |
| 55 | 35 | 10 | Clear stable emulsion |

The mixture containing 30 g GMOI became cloudy upon cooling below 7° C., but the mixture containing 35 g GMOI remained clear to 0° C. The 60:30:10 diesel:GMOI:hydrous ethanol mixture, which contained 0.5 wt % of added water was subjected to water analysis by Karl-Fischer titration (Coffey Laboratories, Inc.) to determine the exact total amount of water present, and a mean result of 1.0 wt %±0.2 wt % was obtained. This indicates that another 0.5 wt % of water was inadvertently introduced by way of water contamination of the GMOI and, to a much lesser extent, of the diesel. This means that the maximum water holding capacity of the 60:30:10 mixture at room temperature is 3.0 wt %. This means that there is considerable flexibility to add water to formulations to enhance NOₓ reduction effects. Further experiments using high purity GMO (90%+) demonstrate that as much as 4% water can be formulated while retaining diesel as the main component.

Experiments to test the sensitivity of emulsions to chemical contaminants were conducted by adding a small amount (10 drops) of concentrated base (50% sodium hydroxide) or concentrated acid (37% hydrochloric acid) to a 60:30:10 emulsion and observing the effects with time. Results showed that the emulsion was quite stable to base, being unchanged after 30 days, but rapidly darkened and separated into two phases after only 4 days upon exposure to acid.

It was found that GMOI samples from different suppliers varied in terms of the minimum amount needed. The cold stability of the emulsions appeared more variable from supplier to supplier. Specifications varied in terms of monooleate content from 40-55%, residual glycerol from 1-3%, and residual triglyceride from 2-5%, but no particular variable clearly correlated with cold stability. In another experiment, a 60:30:10 emulsion with GMOI was stored for 4 days at −15° C., at which point a substantial amount of white flocculent solid had precipitated out. This solid was isolated by vacuum filtration in the cold to give a white waxy solid upon vacuum drying that appeared to be a mixture of trioleate and dioleate by chromatographic analysis. The filtrate obtained, which was now devoid of these solids, remained clear and homogeneous upon prolonged storage at −15° C. It, therefore, appears that the di- and triglyceride contaminants present in formulations using GMOI are a leading cause of solids precipitation in the cold and contribute nothing to the stability of the emulsion because their removal stabilizes rather than destabilizes the emulsion. This evidence provided a strong impetus to seek a means of synthesizing glyceryl monooleate free of di- and trioleate contaminants.

The foregoing formulations are advantageous because they employ only a single surfactant compared to the use of a minimum of two surfactants in prior art examples. The absence of any nitrogen containing substances should help to minimize NOₓ emissions. However, there is considerable product variability depending upon supplier, and the fairly large amounts of GMOI needed rendered the formulation cost about twice that of diesel alone.

### Example 6

### Hydrous Ethanol with Glyceryl Monostearate in Diesel Fuel

30.0 g of Glyceryl monostearate flake was placed in a 250 mL Erlenmeyer flask. 10.0 g of hydrous ethanol (190 proof) was added and stirred. At room temperature, the two did not form a homogeneous solution. Upon gentle warming until the glyceryl monostearate melted (56° C.), the two components mixed to give a homogeneous solution that remained clear upon addition of 60.0 g of diesel fuel while warm. The still warm clear homogeneous emulsion formed a dense white solid precipitate of glyceryl monostearate upon standing at room temperature. Additional experiments using purified GMO having a significant concentration of saturated fats such as stearic acid showed a similar tendency to precipitate solids upon cooling.

### Example 7

### Hydrous Methanol with Industrial GMOI in Diesel Fuel

30.0 g of Industrial GMOI (PPG Industries) was mixed with 10.0 g of anhydrous methanol. 60.0 g of diesel fuel was then added, and the mixture was stirred until clear and homogeneous. 0.5 g of distilled water was added dropwise and stirred until fully dispersed to give a clear homogeneous emulsion of the following final proportions:

| Component | Wt % of Mixture |
|---|---|
| GMOI | 29.8 |
| Hydrous methanol | 10.4 |
| Diesel | 59.8 |

The mixture was clear and stable at room temperature. The phases did not separate after refrigeration for 24 h at 0° C. At room temperature, up to 1 wt % water could be added before phase separation was noted.

Methanol is currently the least expensive of the C₁-C₄ alcohols. Although it is currently manufactured by the reforming of natural gas, it can be produced from synthesic gas obtained by biomass gasification so it has future potential as a renewable energy source.

### Example 8

### C₃ and C₄ Alcohols with GMOI in Diesel Fuel

30.0 g of GMOI (Kemester 2000, 50-60% monoester content) was weighed into each of three flasks. 10.0 g was then added of one of (a) n-propyl alcohol, (b) iso-propyl alcohol, or (c) n-butyl alcohol, and each flask was stirred until a homogeneous mixture was obtained. 60.0 g diesel fuel was then added and thoroughly mixed. In all three cases, homogeneous mixtures were obtained. 0.5 wt % distilled water was then added dropwise to each and mixed until fully dispersed. Again, all three cases gave clear homogeneous mixtures, although it appeared to take longer for the water to disperse in case (c) using n-butyl alcohol. All of the emulsions were stable down to a temperature of 10° C., but a gel-like solid formed upon prolonged storage of samples (a) and (c) at 7° C. The sample using iso-propyl alcohol was stable down to 0° C.

### Example 9

### Hydrous Ethanol with Crude α-GMO in Diesel Fuel

10.0 g of crude α-GMO was placed in a flask. 10.0 g of hydrous ethanol (190 proof) was then added and mixed until homogeneous. 80.0 g of diesel fuel was then added and mixed to give a cloudy suspension. Additional warm liquid α-GMO was added dropwise with stirring until a clear homogeneous mixture was obtained, requiring the addition of 2.7 g. The emulsion was then chilled to 1° C., which resulted in a cloudy emulsion. Addition of a further 0.5 g of α-GMO while still cold rendered a clear emulsion. Further chilling to −13° C. resulted in solids formation and a small amount of a dense liquid phase. Further addition of 1.5 g of α-GMO while in the cold afforded a clear emulsion that was stable to prolonged storage at −13° C. The final proportions needed to achieve stable emulsions over the temperature range are:

| | Wt % of Mixture | | |
|---|---|---|---|
| Component | 20° C. | 1° C. | −13° C. |
| α-GMO | 12.4 | 12.8 | 14.0 |
| Hydrous ethanol | 9.7 | 9.6 | 9.6 |
| Diesel fuel | 77.9 | 77.6 | 76.4 |

The α-GMO used represents a crude synthesis product that was not subjected to any purification. Although the α-GMO was devoid of di- and triglyceride contaminants, there were trace amounts of residual reactants present. Some variability was observed from batch to batch, with the wt % of α-GMO needed to effect a stable emulsion of 10 wt % hydrous2 23° C.:

| Component | Wt % of Mixture |
|---|---|
| Diesel | 57.2-95.2 |
| Hydrous ethanol | 1.9-27.4 |
| α-GMO | 2.9-15.4 |

The emulsion having the maximum ethanol concentration was stable at room temperature, but phase separation occurred upon cooling to 12° C. Upon addition of another 1.7 wt % α-GMO, the emulsion was stable to 0° C. Emulsions having a hydrous ethanol concentration of 10 wt % and 10 wt % crude α-GMO were thermally stable to prolonged storage at −12° C. Note that the α-GMO lot used in this test proved more thermally stable than the lot used in Example 9. There are a number of subtle factors that affect thermal stability. This variability underscores the importance of cleaning up crude α-GMO by solvent partitioning before use.

### Example 11

### Hydrous Ethanol with Commercial 90% GMO in Diesel Fuel

5.0 g of hydrous ethanol and 50.0 g of diesel fuel were added to a flask and mixed. Portions of GMO (Germany, 90% monooleate, M.P. 33-38° C.) were added and mixed until a clear homogeneous mixture was obtained at room temperature. The sample was then chilled to −9° C., at which point a fine white solid and dense liquid phase had formed. Portions of GMO were again added until a mixture was achieved that remained clear and homogeneous at −9° C. The final proportions were:

| | Wt % of Mixture | Wt % of Mixture |
|---|---|---|
| Component | at Room Temp. | at −9° C. |
| Diesel fuel | 86.9 | 86.3 |
| Hydrous ethanol | 8.7 | 8.6 |
| 90% GMO | 4.4 | 5.1 |

This result shows that higher purity GMO grades available commercially are quite suitable as is for producing stable emulsions and can reduce the amount of GMO required by four to six fold over emulsions using industrial GMOI. Although the price for the 90% purity grade is $1.50/lb in bulk versus $0.83/lb for GMOI in bulk , the cost of the emulsion fuel (at a 10% hydrous ethanol level) is $1.66/gallon using high purity GMO versus $2.45/gallon using GMOI. This is quite favorable when compared to the current diesel price of $1.19/gallon. The synthesis and purification methods that are the object of the present invention should enable a reduction in high purity GMO prices by 25-30%, which would render emulsion formulations competitive with diesel.

### Example 12

### Hydrous Ethanol/Diesel Fuel Solubility Over a 0-100% Range Using Commercial 90% GMO

50.00 g of certified diesel (Phillips, Lot D-538) was weighed into a flask, and 5.00 g of hydrous ethanol (190 proof) was added and mixed. GMO (German, 90%) was added in portions and mixed until a clear, homogeneous emulsion was obtained. Another portion of ethanol was added and then more GMO was added to render the mixture clear. These sequential additions were continued until the ethanol concentration exceeded 36 wt %. In a separate experiment, 50.00 g of hydrous ethanol and 5.00 g of certified diesel were weighed into a flask, and GMO was added until clear and homogeneous. This cycle of diesel and GMO additions was continued until the diesel concentration exceeded 36 wt %. Thus, the GMO requirement for blending hydrous ethanol and diesel was determined over the entire range of possible concentrations. These results are tabulated on the following page.

| | Wet | | | | | | |
|---|---|---|---|---|---|---|---|
| | Ethanol | GMO | Diesel | Diesel | Ethanol | Water | GMO |
| | (g) | (g) | (g) | (wt %) | (wt %) | (wt %) | (wt %) |
| Addi- | 5.00 | 3.10 | 50.00 | 86.06 | 8.18 | 0.43 | 5.34 |
| tions | 6.00 | 3.60 | 50.00 | 83.89 | 9.56 | 0.50 | 6.04 |
| to diesel | 7.04 | 4.12 | 50.00 | 81.75 | 10.94 | 0.58 | 6.74 |
| | 8.04 | 4.65 | 50.00 | 79.76 | 12.18 | 0.64 | 7.42 |
| | 9.04 | 5.17 | 50.00 | 77.87 | 13.37 | 0.70 | 8.05 |
| | 10.04 | 5.68 | 50.00 | 76.08 | 14.51 | 0.76 | 8.64 |
| | 11.04 | 6.18 | 50.00 | 74.38 | 15.60 | 0.82 | 9.19 |
| | 12.05 | 6.69 | 50.00 | 72.74 | 16.65 | 0.88 | 9.73 |
| | 13.06 | 7.20 | 50.00 | 71.16 | 17.66 | 0.93 | 10.25 |
| | 14.06 | 7.71 | 50.00 | 69.67 | 18.61 | 0.98 | 10.74 |
| | 15.07 | 8.22 | 50.00 | 68.22 | 19.53 | 1.03 | 11.22 |
| | 16.09 | 8.34 | 50.00 | 67.18 | 20.54 | 1.08 | 11.21 |
| | 17.09 | 8.75 | 50.00 | 65.93 | 21.41 | 1.13 | 11.54 |
| | 18.09 | 9.16 | 50.00 | 64.72 | 22.25 | 1.17 | 11.86 |
| | 19.10 | 9.56 | 50.00 | 63.56 | 23.07 | 1.21 | 12.15 |
| | 21.10 | 10.20 | 50.00 | 61.50 | 24.66 | 1.30 | 12.55 |
| | 22.12 | 10.46 | 50.00 | 60.55 | 25.45 | 1.34 | 12.67 |
| | 23.12 | 10.71 | 50.00 | 59.64 | 26.20 | 1.38 | 12.78 |
| | 24.00 | 11.73 | 50.00 | 58.32 | 26.60 | 1.40 | 13.68 |
| | 28.00 | 13.17 | 50.00 | 54.84 | 29.18 | 1.54 | 14.45 |
| | 30.00 | 14.17 | 50.00 | 53.10 | 30.26 | 1.59 | 15.05 |
| | 35.00 | 15.42 | 50.00 | 49.79 | 33.11 | 1.74 | 15.36 |
| | 37.00 | 16.42 | 50.00 | 48.35 | 33.99 | 1.79 | 15.88 |
| | 42.00 | 17.67 | 50.00 | 45.59 | 36.38 | 1.91 | 16.11 |
| Addi- | 50.00 | 17.37 | 36.45 | 35.11 | 45.75 | 2.41 | 16.73 |
| tions | 50.00 | 15.91 | 31.00 | 31.99 | 49.01 | 2.58 | 16.42 |
| to | 50.00 | 14.21 | 25.49 | 28.42 | 52.95 | 2.79 | 15.84 |
| Ethanol | 50.00 | 12.25 | 20.04 | 24.35 | 57.72 | 3.04 | 14.89 |
| | 50.00 | 10.01 | 15.03 | 20.03 | 63.30 | 3.33 | 13.34 |
| | 50.00 | 8.50 | 12.53 | 17.64 | 66.87 | 3.52 | 11.97 |
| | 50.00 | 6.88 | 10.03 | 14.99 | 70.99 | 3.74 | 10.28 |
| | 50.00 | 2.31 | 7.50 | 12.54 | 79.42 | 4.18 | 3.86 |
| | 50.00 | 1.04 | 5.00 | 8.92 | 84.76 | 4.46 | 1.86 |

### Example 13

### 90% GMO Requirements for Emulsions with High Water and Ethanol Concentrations

40.00 g of certified diesel (Phillips, Lot D-538) and 24.00 g of anhydrous ethanol were weighed into a flask. A portion of water was then added, followed by portions of GMO (German, 90%) until a clear, homogeneous emulsion was obtained. This cycle of water and GMO additions was continued until the water concentration exceeded 5 wt % with the following results:

Composition of Concentrated Stable
Formulations of Water and Ethanol with 90% GMO

| Diesel (wt %) | Ethanol (wt %) | Water (wt %) | GMO (wt %) |
|---|---|---|---|
| 62.51 | 37.49 | 0.00 | 0.00 |
| 58.81 | 35.28 | 1.50 | 4.41 |
| 54.78 | 32.86 | 2.77 | 9.60 |
| 52.62 | 31.57 | 4.00 | 11.81 |
| 51.14 | 30.67 | 5.11 | 13.08 |

### Example 14

### 90% GMO Requirements for Emulsions with High Water and Low Ethanol Concentrations

25.00 g of No. 2 diesel fuel and 0.51 g of distilled water were measured into a flask, and portions of GMO (German, 90%) were added and mixed until dispersed. After addition of 1.72 g GMO, it was evident that the water and GMO were not going to mix to give a clear emulsion, although the water was well-dispersed. Upon addition of 1.00 g of anhydrous ethanol, the mixture formed a clear emulsion, which became cloudy upon further addition of 0.72 g of anhydrous ethanol. Addition of a 0.28 g portion of GMO again afforded a clear emulsion. Portions of water were then added followed by portions of GMO until clear emulsions were obtained. The proportions affording clear, stable emulsions at room temperature are summarized in the following table:

| Wet | | | | | | | |
|---|---|---|---|---|---|---|---|
| Ethanol | Water | GMO | Diesel | Diesel | Ethanol | Water | GMO |
| (g) | (g) | (g) | (g) | (wt %) | (wt %) | (wt %) | (wt %) |
| 1.00 | 0.51 | 1.72 | 25.00 | 88.56 | 3.54 | 1.81 | 6.09 |
| 1.78 | 0.51 | 2.00 | 25.00 | 85.35 | 6.08 | 1.74 | 6.83 |
| 1.78 | 0.64 | 2.31 | 25.00 | 84.09 | 5.99 | 2.15 | 7.77 |
| 1.78 | 0.72 | 2.50 | 25.00 | 83.33 | 5.93 | 2.40 | 8.33 |
| 1.78 | 1.02 | 3.90 | 25.00 | 78.86 | 5.62 | 3.22 | 12.30 |
| 1.78 | 1.37 | 6.36 | 25.00 | 72.44 | 5.16 | 3.97 | 18.43 |
| 1.78 | 1.59 | 8.37 | 25.00 | 68.05 | 4.84 | 4.33 | 22.78 |
| 1.78 | 1.93 | 10.46 | 25.00 | 63.82 | 4.54 | 4.93 | 26.70 |
| 1.78 | 2.26 | 12.76 | 25.00 | 59.81 | 4.26 | 5.41 | 30.53 |
| 1.78 | 2.66 | 15.06 | 25.00 | 56.18 | 4.00 | 5.98 | 33.84 |
| 1.78 | 3.06 | 17.20 | 25.00 | 53.15 | 3.78 | 6.51 | 36.56 |
| 1.78 | 3.41 | 19.42 | 25.00 | 50.39 | 3.59 | 6.87 | 39.15 |
| 1.78 | 3.78 | 21.79 | 25.00 | 47.76 | 3.40 | 7.22 | 41.62 |

### Example 15

### Hydrous Ethanol with the Ammonium Salt of Oleic Acid and Crude α-GMO in Diesel Fuel

5.0 g of oleic acid was first mixed with 6.0 g of hydrous ethanol (190 proof) in a flask. 0.55 g of 28% aqueous ammonium hydroxide was then added and mixed until a clear homogeneous solution was obtained. 22.5 g of diesel fuel was then added in portions and mixed to the following final proportions:

| Component | Wt % of Mixture |
|---|---|
| Diesel | 66.1 |
| Hydrous ethanol | 17.6 |
| Oleic acid | 14.7 |
| Ammonium hydroxide | 1.6 |

The resulting emulsion was clear and homogeneous at 23° C.

Upon addition of 27.5 g more diesel fuel, phase separation occurred. Crude α-GMO obtained by direct synthesis was then added in portions until a clear homogeneous emulsion was obtained with the following final proportions:

| Component | Wt % of Mixture |
|---|---|
| Diesel | 78.7 |
| Hydrous ethanol | 9.4 |
| Oleic acid | 7.9 |
| Ammonium hydroxide | 0.9 |
| α-GMO | 3.1 |

The emulsion was stable at 23° C. Upon cooling to 12° C., phase separation occurred. Upon addition of 0.5 wt % α-GMO, the emulsion cleared and remained stable to 0° C. Results of emissions tests shown in the table following Example 16 show a dramatic drop in NOₓ emissions using the formulation with ammonia even though cetane enhancer is absent. The same formulation without ammonia showed no reduction in emissions. This is strong evidence that ammonia is exerting a “neutralizing” effect on NOₓ, presumably by reacting with NOₓ to give nitrogen and water.

### Example 16

### Formulations Including Cetane Enhancers

Two emulsions were formulated by successive mixing of ingredients in a flask to the following final compositions:

| Component | Composition 1 (wt %) | Composition 2 (wt %) |
|---|---|---|
| Certified Diesel | 73 | 51 |
| Ethanol (Anhydrous) | 12 | 24 |
| Water | 2 | 4 |
| GMO (German, 90%) | 13 | 18 |

The cetane enhancer, 2-ethylhexyl nitrate, was added in proportions of 1.5 and 3 wt % to compositions 1 and 2, respectively. The properties of the resulting emulsions were compared to the original emulsions lacking cetane enhancer. All compositions remained stable to temperatures down to −8° C., although those having cetane enhancer appeared to be somewhat more stable to colder temperatures. The cetane number of composition 1 was raised from 37.8 to 51.1 by the addition of 1.5 wt % of 2-ethylhexyl nitrate.

Selected formulations were tested for cetane number, exhaust emissions, and mileage at California Environmental Engineering using a 1995 Dodge Ram and certified testing procedures. The results are presented in the following table:

EMULSION FUEL DIESEL ENGINE TEST RESULTS
(Conducted by CEE 3-5/99, ′95 Dodge Ram)

| | | Emissions (grams/mile) | | | | | |
|---|---|---|---|---|---|---|---|
| | | HC | | | | | Mile- |
| | Cetane | Partic- | | | | | age |
| Formulation | Number | ulates | CO | | NOx | CO2 | (MPG) |
| Pure diesel | 46.7 | 0.51 | 1.91 | 6.83 | 555.77 | 0.16 | 18.16 |
| Example 16 | 51.1 | 0.87 | 1.66 | 6.26 | 580.21 | 0.10 | 17.38 |
| Comp. 1 | | | | | | | |
| Example 16 | 46.7 | 0.81 | 1.80 | 6.06 | 572.37 | 0.13 | 17.61 |
| Comp. 2 | | | | | | | |
| Example 15 | N.D. | 0.78 | 1.94 | 6.16 | 581.62 | 0.17 | 17.33 |
| Example 18 | N.D. | 0.12 | 1.64 | 5.95 | 492.51 | 0.07 | 20.50 |

HC = Hydrocarbons
NOx = Nitrogen Oxides
NH3 = Ammonia
CO = Carbon Monoxide
CO2 = Carbon Dioxide

The test vehicle employed represents a late model vehicle that produces inherently lower emissions than earlier model or heavy-duty engines. Although a number of formulations afforded emissions reductions when tested in an earlier model engine (1989 Cummins), the same formulations afforded little or no emissions reductions when re-tested in the 1995 vehicle. Both compositions referenced in Example 16 afforded dramatic reductions in NOₓ and particulate, while fuel economy was maintained. Examination of particulate filters used in these tests shows very low soot levels. The cetane enhancer proved important in realizing emissions reduction by reducing the ignition time of the fuel which would otherwise be retarded by the presence of water and ethanol. As noted, Example 15 containing ammonia also showed a dramatic reduction in NOₓ emissions, even though one would predict no emissions reduction because cetane enhancer was absent. Indeed, there was no reduction in particulate emissions as expected. This powerful NOₓ reducing effect of ammonia is in addition to the emissions reducing effects of water and ethanol in the presence of cetane enhancer. Thus, formulations having both water, ethanol, ammonia, and cetane enhancer are predicted to give twice the NOₓ emissions reduction shown for either separately.

### Example 17

### Formulations of 90% GMO With Ethylene Glycol

10.0 g of certified diesel and 1.0 g of ethylene glycol were added to a flask and mixed. GMO (German, 90%) was added in portions and mixed until a clear, homogeneous emulsion was obtained with the following final composition:

| Component | Wt % of Mixture |
|---|---|
| Diesel | 69.0 |
| Ethylene glycol | 6.9 |
| GMO | 24.1 |

Although stable at room temperature, chilling quickly induced the precipitation of white solids.

### Example 18

### Formulations with Ammonia and Cetane Enhancer

71.35 g of certified diesel fuel (lot D-538) was weighed into a flask. 12.63 g of hydrous ethanol (190 proof, USP grade) was added and mixed to give two immiscible phases. 10.62 g of oleic acid (USP grade) was added and mixed to give a very hazy unstable suspension. 190 g of 28% ammonium hydroxide solution (technical grade) was added and mixed to give a clear homogeneous emulsion. 1.50 g of 2-ethyl hexyl nitrate was added and mixed. The emulsion was stable at room temperature but became very cloudy upon cooling to 0° C. Portions of high purity GMO (German) were added and mixed until the resulting emulsion was stable overnight at −14° C. 2.00 g of GMO was required. The final proportions required to produce stable emulsions at various temperatures are shown in the following table:

Wt % of Mixture

| Component | 21° C. | 0° C. | −14° C. |
|---|---|---|---|
| Diesel | 72.8 | 72.1 | 71.4 |
| Ethanol | 12.2 | 12.1 | 12.0 |
| Water | 2.0 | 2.0 | 2.0 |
| Ammonia | 0.5 | 0.5 | 0.5 |
| 2-Ethyl hexyl nitrate | 1.5 | 1.5 | 1.5 |
| Oleic acid | 10.8 | 10.7 | 10.6 |
| High purity GMO | 0.0 | 1.0 | 2.0 |

Results of emissions tests shown in the table following Example 16 show a dramatic drop in NOₓ emissions using the formulation with ammonia. The same formulation without ammonia showed a smaller reduction in NOₓ and particulate emissions. This provides additional confirmation that ammonia is exerting a “neutralizing” effect on NOₓ.

### Low Water Microemulsions

A low-water microemulsion containing approximately 2 wt % water rather than relatively high-water microemulsions containing approximately 10 wt % water have also been found to be effective to reduce undesired emissions and to provide desirable mileage and performance. By contrast, a relatively high-water microemulsion shows increased emissions reductions but also exhibits a 15% loss in mileage and peak horsepower (see table):

Percent
Reduction

| Sample | NOx | PM |
|---|---|---|
| 2% Water | 8.4 | 38.2 |
| 10% | 12.7 | 57.3 |
| Water | | |

The low water formulation shown below was produced to examine its cold temperature stability. The formulation had the following proportions:

| Component | wt % (gms) |
|---|---|
| Diesel | 73.1 |
| Ethanol 95% | 12.5 |
| Oleic acid | 10.9 |
| Ammonia 28% aqueous | 1.9 |
| 2-EHN | 1.5 |
| Water | None added since there was sufficient water |
| | introduced via the ammonia (1.36 gm) and the |
| | 95% ethanol (0.61 gm) |

The above formulation was stable at room temperature and above but immediately separated into two phases below 60° F.

It is also desirable to reduce the ethanol content to raise the flashpoint of the formulation to within the diesel range. The following formulation was prepared with 8% ethanol rather than 12% ethanol. This formulation had the following composition:

| Component | wt % (gms) |
|---|---|
| Diesel | 76.6 |
| Ethanol 95% | 8.4 |
| Oleic acid | 11.4 |
| Ammonia 28% aqueous | 2.0 |
| 2-EHN | 1.6 |
| Water | None added since there was sufficient water |
| | introduced via the ammonia (1.36 gm) and the |
| | 95% ethanol (0.61 gm) |

The above microemulsion was extremely stable to the cold remaining clear down to 6.4° F. and slightly viscous but unbroken down to 1.1° F.

In the preceding description, various aspects of claimed subject matter have been described. For purposes of explanation, specific numbers, systems and/or configurations were set forth to provide a thorough understanding of the claimed subject matter. However, it should be apparent to one skilled in the art having the benefit of this disclosure that claimed subject matter may be practiced without the specific details. In other instances, features that would be understood by one of ordinary skill were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all such modifications and/or changes as fall within the true spirit of claimed subject matter.

The invention can also be described in the following paragraphs:

### I. Fuel/Biodiesel/Diesel Compositions.

A/0. A fuel composition for use in internal-combustion engines, the fuel composition comprising:

a fuel component;

an alcohol component;

a water component;

an anhydrous emulsifier component chosen from the group consisting of glyceryl monooleate and ammonium oleate, being present in an amount effective for the fuel, alcohol, water, and emulsifier components to form an emulsion; and

a cetane-enhancer component.

A/1. The fuel composition of paragraph NO, wherein the alcohol has one to four carbons.
A/2. The fuel composition of paragraph A/1, wherein the alcohol is selected from the group consisting of methanol and ethanol.
A/3. The fuel composition of paragraph A/0, wherein the alcohol is aqueous alcohol.
A/4. The fuel composition of paragraph A/0, wherein the fuel, alcohol, water, and emulsifier components are present in amounts effective to reduce emissions of nitrogen oxides by a threshold amount upon combustion of the fuel composition, relative to fuel alone.
A/5. The fuel composition of paragraph A/0, wherein the fuel, alcohol, water, and emulsifier components are present in amounts effective to reduce emissions of particulates by a threshold amount upon combustion of the fuel composition, relative to fuel alone.
A/6. The fuel composition of paragraph A/5, wherein the fuel, alcohol, water, and emulsifier components also are present in amounts effective to reduce emissions of nitrogen oxides by a threshold amount upon combustion of the fuel composition, relative to fuel alone.
A/7. The fuel composition of paragraph A/6, wherein the polyol fatty acid esters and polyol fatty alcohol ethers include monoglycerides of unsaturated fatty acids.

A/8. The fuel composition of paragraph A/7, wherein the unsaturated fatty acids are selected from the group consisting of arachidic, elaidic acid, erucic acid, gadoleic acid, margaroleic acid, myristoleic acid, linoleic acid, linolenic acid, palmitoleic acid and oleic acid.

A/9. The fuel composition of paragraph A/8, wherein the unsaturated fatty acid is oleic acid.
A/10. The fuel composition of paragraph A/9, wherein at least about half of the monoglycerides are end substituted.

A/11. The fuel composition of paragraph A/7, wherein at least about half of the monoglycerides are end substituted.

A/12. The fuel composition of paragraph A/0, wherein the fuel, alcohol, water, and emulsifier components are present in amounts effective to form an emulsion containing no more than about 90% fuel.
A/13. The fuel composition of paragraph A/0, wherein the fuel, alcohol, water, and emulsifier components are present in amounts effective to form micelles in which a substantially monomolecular layer of emulsifier component surrounds a substantially alcohol/water core.
A/14. The fuel composition of paragraph A/0, wherein the fuel composition is fluid at room temperature.
A/15. The fuel composition of paragraph A/0, wherein the fuel composition is suspended in air to form an aerosol.
A/16. The fuel composition of paragraph A/0, wherein the fuel composition has a viscosity of less than about 10 millipascals.
A/17. The fuel composition of paragraph A/0, further comprising a small amount of one or more additives selected from the following group: thermal stabilizers, and combustion modifiers.
A/18. The fuel composition of paragraph A/0, wherein the emulsifier component consists essentially of a single species selected from the group consisting of noncyclic polyol fatty acid esters and noncyclic polyol fatty alcohol ethers.
A/19. The fuel composition of paragraph A/0, wherein the emulsifier component is made substantially of glyceryl-α-monooleate.
A/20. The fuel composition of paragraph A/1, wherein the alcohol is selected from the group consisting of envirolene, n-butanol and isopropanol.
B/0. A biodiesel composition for use in internal-combustion engines, the biodiesel composition comprising:

a biodiesel fuel component;

an alcohol component;

a water component;

an anhydrous emulsifier component chosen from the group consisting of glyceryl monooleate and ammonium oleate, being present in an amount effective for the biodiesel fuel, alcohol, water, and emulsifier components to form an emulsion; and

a cetane-enhancer component.

B/1. The biodiesel composition of paragraph B/O, wherein the alcohol has one to four carbons.
B/2. The biodiesel composition of paragraph B/1, wherein the alcohol is selected from the group consisting of methanol and ethanol.
B/3. The biodiesel composition of paragraph B/0, wherein the alcohol is aqueous alcohol.
B/4. The biodiesel composition of paragraph B/0, wherein the biodiesel fuel, alcohol, water, and emulsifier components are present in amounts effective to reduce emissions of nitrogen oxides by a threshold amount upon combustion of the biodiesel composition, relative to biodiesel fuel alone.
B/5. The biodiesel composition of paragraph B/0, wherein the biodiesel fuel, alcohol, water, and emulsifier components are present in amounts effective to reduce emissions of particulates by a threshold amount upon combustion of the biodiesel composition, relative to biodiesel fuel alone.
B/6. The biodiesel composition of paragraph B/5, wherein the biodiesel fuel, alcohol, water, and emulsifier components also are present in amounts effective to reduce emissions of nitrogen oxides by a threshold amount upon combustion of the biodiesel composition, relative to biodiesel fuel alone.
B/7. The biodiesel composition of paragraph B/6, wherein the polyol fatty acid esters and polyol fatty alcohol ethers include monoglycerides of unsaturated fatty acids.
B/8. The biodiesel composition of paragraph B/7, wherein the unsaturated fatty acids are selected from the group consisting of arachidic, elaidic acid, erucic acid, gadoleic acid, margaroleic acid, myristoleic acid, linoleic acid, linolenic acid, palmitoleic acid and oleic acid.
B/9. The biodiesel composition of paragraph B/8, wherein the unsaturated fatty acid is oleic acid.
B/10. The biodiesel composition of paragraph B/9, wherein at least about half of the monoglycerides are end substituted.
B/11. The biodiesel composition of paragraph B/7, wherein at least about half of the monoglycerides are end substituted.
B/12. The biodiesel composition of paragraph B/0, wherein the biodiesel fuel, alcohol, water, and emulsifier components are present in amounts effective to form an emulsion containing no more than about 90% biodiesel fuel.
B/13. The biodiesel composition of paragraph B/0, wherein the biodiesel fuel, alcohol, water, and emulsifier components are present in amounts effective to form micelles in which a substantially monomolecular layer of emulsifier component surrounds a substantially alcohol/water core.
B/14. The biodiesel composition of paragraph B/0, wherein the biodiesel composition is fluid at room temperature.
B/15. The biodiesel composition of paragraph B/0, wherein the biodiesel composition is suspended in air to form an aerosol.
B/16. The biodiesel composition of paragraph B/0, wherein the biodiesel composition has a viscosity of less than about 10 millipascals.
B/17. The biodiesel composition of paragraph B/0, further comprising a small amount of one or more additives selected from the following group: thermal stabilizers, and combustion modifiers.
B/18. The biodiesel composition of paragraph B/0, wherein the emulsifier component consists essentially of a single species selected from the group consisting of noncyclic polyol fatty acid esters and noncyclic polyol fatty alcohol ethers.
B/19. The biodiesel composition of paragraph B/0, wherein the emulsifier component is made substantially of glyceryl-α-monooleate.
B/20. The biodiesel composition of paragraph B/1, wherein the alcohol is selected from the group consisting of envirolene, n-butanol and isopropanol.
C/0. A diesel composition for use in internal-combustion engines, the diesel composition comprising:

a diesel fuel component;

an alcohol component;

a water component;

an anhydrous emulsifier component chosen from the group consisting of glyceryl monooleate and ammonium oleate, being present in an amount effective for the diesel fuel, alcohol, water, and emulsifier components to form an emulsion; and

a cetane-enhancer component.

C/1. The diesel composition of paragraph C/0, wherein the alcohol has one to four carbons.
C/2. The diesel composition of paragraph C/1, wherein the alcohol is selected from the group consisting of methanol and ethanol.
C/3. The diesel composition of paragraph C/0, wherein the alcohol is aqueous alcohol.
C/4. The diesel composition of paragraph C/0, wherein the diesel fuel, alcohol, water, and emulsifier components are present in amounts effective to reduce emissions of nitrogen oxides by a threshold amount upon combustion of the diesel composition, relative to diesel fuel alone.
C/5. The diesel composition of paragraph C/0, wherein the diesel fuel, alcohol, water, and emulsifier components are present in amounts effective to reduce emissions of particulates by a threshold amount upon combustion of the diesel composition, relative to diesel fuel alone.
C/6. The diesel composition of paragraph C/5, wherein the diesel fuel, alcohol, water, and emulsifier components also are present in amounts effective to reduce emissions of nitrogen oxides by a threshold amount upon combustion of the diesel composition, relative to diesel fuel alone.
C/7. The diesel composition of paragraph C/6, wherein the polyol fatty acid esters and polyol fatty alcohol ethers include monoglycerides of unsaturated fatty acids.
C/8. The diesel composition of paragraph C/7, wherein the unsaturated fatty acids are selected from the group consisting of arachidic, elaidic acid, erucic acid, gadoleic acid, margaroleic acid, myristoleic acid, linoleic acid, linolenic acid, palmitoleic acid and oleic acid.
C/9. The diesel composition of paragraph C/8, wherein the unsaturated fatty acid is oleic acid.
C/10. The diesel composition of paragraph C/9, wherein at least about half of the monoglycerides are end substituted.
C/11. The diesel composition of paragraph C/7, wherein at least about half of the monoglycerides are end substituted.
C/12. The diesel composition of paragraph C/0, wherein the diesel fuel, alcohol, water, and emulsifier components are present in amounts effective to form an emulsion containing no more than about 90% diesel fuel.
C/13. The diesel composition of paragraph C/0, wherein the diesel fuel, alcohol, water, and emulsifier components are present in amounts effective to form micelles in which a substantially monomolecular layer of emulsifier component surrounds a substantially alcohol/water core.
C/14. The diesel composition of paragraph C/0, wherein the diesel composition is fluid at room temperature.
C/15. The diesel composition of paragraph C/0, wherein the diesel composition is suspended in air to form an aerosol.
C/16. The diesel composition of paragraph C/0, wherein the diesel composition has a viscosity of less than about 10 millipascals.
C/17. The diesel composition of paragraph C/0, further comprising a small amount of one or more additives selected from the following group: thermal stabilizers, and combustion modifiers.
C/18. The diesel composition of paragraph C/0, wherein the emulsifier component consists essentially of a single species selected from the group consisting of noncyclic polyol fatty acid esters and noncyclic polyol fatty alcohol ethers.
C/19. The diesel composition of paragraph C/0, wherein the emulsifier component is made substantially of glyceryl-α-monooleate.
C/20. The diesel composition of paragraph C/1, wherein the alcohol is selected from the group consisting of envirolene, n-butanol and isopropanol.
D/0. A fuel composition for use in internal-combustion engines, the fuel composition comprising:

a hydrocarbon fuel component suitable for use in internal-combustion engines;

an alcohol component;

a water component;

an anhydrous emulsifier component; and

wherein the water and emulsifier are each provided in preselected amounts that are effective to achieve the desired NOX reduction, and wherein the water is provided in an amount according to the following formula: x % water to produce the desired NOX reduction in the range of 3-4x.

D/1. The fuel composition of paragraph D/0, wherein the hydrocarbon fuel component is substantially biodiesel fuel.
D/2. The fuel composition of paragraph D/0, wherein at least about half of the portion is end-substituted.
D/3. The fuel composition of paragraph D/0, wherein at least about half of the portion is a single species.
E/0. A fuel composition for use in internal-combustion engines, the fuel composition comprising:

a hydrocarbon fuel component suitable for use in internal-combustion engines;

a water component;

an emulsifier component, the emulsifier being present in an amount effective for the hydrocarbon fuel, polar fluid, and emulsifier components to form an emulsion,

wherein the emulsifier consists essentially of purified glyceryl monooleate; and

a cetane-enhancer component.

F/0. A fuel composition for use in internal-combustion engines, the fuel composition comprising:

a hydrocarbon fuel component suitable for use in internal-combustion engines;

an alcohol component;

a water component; and

an emulsifier component consisting essentially of a single molecular species having a polar portion and a nonpolar portion, the emulsifier being present in an amount effective for the hydrocarbon fuel, alcohol, water, and emulsifier to form micelles containing the alcohol, water, and emulsifier.

F/1. The fuel composition of paragraph F/0, wherein the emulsifier component comprises a combination of a polyol fatty acid ester or polyol fatty alcohol ether with the ammonium salt of a carboxylic acid.
F/2. The fuel composition of paragraph F/1, wherein the carboxylic acid is oleic acid.
G/0. A biodiesel composition for use in internal-combustion engines, the biodiesel composition consisting essentially of:

biodiesel fuel;

a polar fluid; and

an emulsifier, the emulsifier being present in an amount effective for the hydrocarbon fuel, polar fluid, and emulsifier to form an emulsion,

wherein at least a portion of the emulsifier is selected from the group consisting essentially of noncyclic polyol fatty acid esters and noncyclic polyol fatty alcohol ethers, at least about half of that portion being mono-substituted.

G/1. The biodiesel composition of paragraph G/0, wherein the polar fluid includes oxygen.
G/2. The biodiesel composition of paragraph G/0, wherein the polar fluid is selected from the group consisting of water and alcohol.
H/0. A biodiesel composition for use in internal-combustion engines, the biodiesel composition comprising:

biodiesel fuel; and

an oxygen-containing fluid;

wherein the oxygen-containing fluid is substantially homogeneously dispersed in the biodiesel to form the biodiesel composition.

H/1. The biodiesel composition of paragraph H/0, further comprising an emulsifier, the emulsifier being present in an amount effective for the biodiesel fuel, oxygen-containing fluid, and emulsifier to form an emulsion.

### II. Methods of Making Fuel Compositions

A/0. A method of formulating a fuel composition for use in internal-combustion engines, the method comprising:

providing a fuel; and

forming the fuel composition by adding to the fuel a polar fluid and an emulsifier, the emulsifier being present in an amount effective for the biodiesel fuel, alcohol, water, and emulsifier to form an emulsion, and wherein the emulsifier includes an active surfactant mixture of oleic acid and ammonium oleate, wherein the ratio of oleic acid to ammonium oleate is no more than 60:40.

A/1. The method of paragraph A/0, further comprising mixing the fuel composition with air to form an aerosol.
A/2. The method of paragraph A/1, further comprising combusting the fuel composition.
A/3. The method of paragraph A/0, further comprising requiring the emulsifier to include purified glyceryl monooleate.
A/4. The method of paragraph A/0, further comprising requiring the ration of active surfactant mixture of oleic acid and ammonium oleate to be substantially 50:50.
B/0. A method involving forming a polyol fatty acid ester, the method comprising:

providing a polyol having at least three reactive alcohol groups;

selecting a fatty acid;

protecting all but a preselected one of the reactive alcohol groups on the polyol by reacting all but the preselected one of the reactive alcohol groups with protecting groups, wherein a single protecting group may protect one or more reactive alcohol groups;

linking the fatty acid to the polyol through an ester linkage by reacting the fatty acid with the preselected one of the reactive alcohol groups; and

forming the polyol fatty acid ester by removing the protecting groups;

wherein each of the steps is performed under conditions that would tend not to substantially reduce an unsaturated fatty acid.

B/1. The method of paragraph B/0, wherein the fatty acid is unsaturated.
B/2. The method of paragraph 40, wherein the fatty acid is selected from the group consisting of arachidic, elaidic acid, erucic acid, gadoleic acid, margaroleic acid, myristoleic acid, linoleic acid, linolenic acid, palmitoleic acid and oleic acid.
B/3. The method of paragraph B/0, wherein the polyol is a noncyclic polyol.
B/4. The method of paragraph B/0, wherein the polyol is glycerol.
B/5. The method of paragraph B/0, wherein the protecting group is acetone.
B/6. The method of paragraph B/0, wherein the protecting group is benzaldehyde.
B/7. The method of paragraph B/0, wherein the step of removing the protecting group includes acid hydrolysis.
B/8. The method of paragraph B/0, wherein the step of removing the protecting group includes formation and subsequent hydrolysis of a borate ester.
B/9. The method of paragraph B/7, wherein the acid hydrolysis is performed using acetic acid.
B/10. The method of paragraph B/0, wherein each of the steps is performed under an inert atmosphere.
B/11. The method of paragraph B/10, wherein the inert atmosphere consists essentially of nitrogen.
B/12. The method of paragraph B/0, wherein each of the steps is performed in the same reaction vessel.
B/13. The method of paragraph B/0, further comprising purifying the fatty acid ester.
B/14. The method of paragraph B/0, further comprising combining the fatty acid ester with a hydrocarbon fuel and a polar fluid to form an emulsion suitable for use as a fuel composition in an internal-combustion engine.
B/15. The method of paragraph B/0, further comprising linking an additional moiety to a second reactive alcohol group different than the preselected one of the reactive alcohol groups.
B/16. The method of paragraph B/15, wherein the additional moiety nitrates one or both hydroxyl groups on the polyol fatty acid ester to produce a nitro polyol fatty acid ester.
B/17. The method of paragraph B/16, wherein the nitro polyol fatty acid ester is a cetane enhancer.
B/18. The method of paragraph B/17, wherein the cetane enhancer retains at least a portion of its surfactant properties.
B/19. The method of paragraph B15, wherein the additional moiety is ammonia and the polyol fatty acid ester is glyceryl monooleate
C/20. A method involving forming a polyol fatty acid ester, the method comprising:

providing a polyol having at least three reactive alcohol groups;

selecting a fatty acid chloride;

protecting all but a preselected one of the reactive alcohol groups on the polyol by reacting all but the preselected one of the reactive alcohol groups with protecting groups, wherein a single protecting group may protect one or more reactive alcohol groups;

linking the fatty acid chloride to the polyol through an ester linkage by reacting the fatty acid with the preselected one of the reactive alcohol groups in the presence of an acid scavenger; and

forming the polyol fatty acid ester by removing the protecting groups;

wherein each of the steps is performed under conditions that would tend not to substantially reduce an unsaturated fatty acid.

C/21. The method of paragraph C/20, wherein the acid scavenger is pyridine.
D/0. A method involving separating monoglycerides from a mixture containing monoglycerides and multiglycerides, the method comprising:

providing a mixture of monoglycerides and multiglycerides;

selecting an extraction fluid having a nonpolar component and a polar component that includes aqueous ethanol;

preferentially associating the monoglycerides with the polar component and the multiglycerides with the nonpolar component by contacting the mixture with the extraction fluid; and

at least partially separating the monoglycerides from the multiglycerides by separating the polar component from the nonpolar component.

D/1. The method of paragraph D/0, wherein the monoglycerides and multiglycerides primarily include esters of oleic acid.
D/2. The method of paragraph D/0, wherein the polar component consists essentially of aqueous ethanol.
D/3. The method of paragraph D/2, wherein the aqueous ethanol includes about five percent water.
D/4. The method of paragraph D/0, wherein the nonpolar component includes hexane.
D/5. The method of paragraph D/4, wherein the polar component consists essentially of aqueous ethanol.
D/6. The method of paragraph D/5, wherein the extraction fluid includes about fifteen parts of the polar component for every ten parts of the nonpolar component.
D/7. The method of paragraph D/0, wherein the polar component is separated from the nonpolar component by contacting the two components with water.
D/8. The method of paragraph D/0, further comprising concentrating the polar component to concentrate the monoglycerides.
D/9. The method of paragraph D/8, further comprising combining the concentrated polar component with a hydrocarbon fuel and a polar fluid to form an emulsion suitable for use as a fuel composition in an internal-combustion engine.
D/10. The method of paragraph D/9, further comprising repeating the steps of preferentially associating the monoglycerides and separating the monoglycerides, using the ethanol component as the mixture.
D/11. The method of paragraph D/10, wherein the step of contacting the mixture with an extraction fluid includes using a counter-current process.
D/12. The method of paragraph D/11, wherein the counter-current process includes using the polar component in a descending phase and the nonpolar component in an ascending phase.
D/13. The method of paragraph D/0, further comprising using the monoglycerides as an emulsifier in a fuel composition for an internal-combustion engine.
D/14. The method of paragraph D/0, wherein the polar component and the nonpolar component form distinct phases.
E/0. A method involving separating polyol monoesters from a mixture of polyol monoesters and polyol multi-esters, the polyol including at least a four-carbon chain, the method comprising:

providing a mixture of polyol monoesters and polyol multi-esters;

selecting an extraction fluid having a nonpolar component and a polar component;

preferentially associating the polyol monoesters with the polar component and the polyol multi-esters with the nonpolar component by contacting the mixture with the extraction fluid; and

at least partially separating the polyol monoesters from the polyol multi-esters by separating the polar component from the nonpolar component.

I claim:

1. A method of formulating a fuel composition for use in internal-combustion engines, the method comprising:
providing a fuel; and
forming the fuel composition by adding to the fuel a polar fluid and an emulsifier, the emulsifier being present in an amount effective for the biodiesel fuel, alcohol, water, and emulsifier to form an emulsion, and wherein the emulsifier includes an active surfactant mixture of oleic acid and ammonium oleate, wherein the ratio of oleic acid to ammonium oleate is no more than 60:40.

2. The method of claim 1, further comprising mixing the fuel composition with air to form an aerosol.

3. The method of claim 2, further comprising combusting the fuel composition.

4. The method of claim 1, further comprising requiring the emulsifier to include purified glyceryl monooleate.

5. The method of claim 1, further comprising requiring the ration of active surfactant mixture of oleic acid and ammonium oleate to be substantially 50:50.

6. A method involving forming a polyol fatty acid ester, the method comprising:
providing a polyol having at least three reactive alcohol groups;
selecting a fatty acid;
protecting all but a preselected one of the reactive alcohol groups on the polyol by reacting all but the preselected one of the reactive alcohol groups with protecting groups, wherein a single protecting group may protect one or more reactive alcohol groups;
linking the fatty acid to the polyol through an ester linkage by reacting the fatty acid with the preselected one of the reactive alcohol groups; and
forming the polyol fatty acid ester by removing the protecting groups;
wherein each of the steps is performed under conditions that would tend not to substantially reduce an unsaturated fatty acid.

7. The method of claim 6, wherein the fatty acid is unsaturated.

8. The method of claim 6, wherein the fatty acid is selected from the group consisting of arachidic, elaidic acid, erucic acid, gadoleic acid, margaroleic acid, myristoleic acid, linoleic acid, linolenic acid, palmitoleic acid and oleic acid.

9. The method of claim 6, wherein the polyol is a noncyclic polyol.

10. The method of claim 6, wherein the polyol is glycerol.

11. The method of claim 6, wherein the protecting group is acetone.

12. The method of claim 6, wherein the protecting group is benzaldehyde.

13. The method of claim 6, wherein the step of removing the protecting group includes acid hydrolysis.

14. The method of claim 6, wherein the step of removing the protecting group includes formation and subsequent hydrolysis of a borate ester.

15. The method of claim 13, wherein the acid hydrolysis is performed using acetic acid.

16. The method of claim 6, wherein each of the steps is performed under an inert atmosphere.

17. The method of claim 16, wherein the inert atmosphere consists essentially of nitrogen.

18. The method of claim 6, wherein each of the steps is performed in the same reaction vessel.

19. The method of claim 6, further comprising purifying the fatty acid ester.

20. The method of claim 6, further comprising combining the fatty acid ester with a hydrocarbon fuel and a polar fluid to form an emulsion suitable for use as a fuel composition in an internal-combustion engine.
